(19) United States
(12) Patent Application Publication
(10) Pub. No.: US 2016/0113904 A1
(43) Pub. Date: Apr. 28, 2016

(54) SKIN CARE PRODUCTS COMPRISING SUBSTITUTED HYDROXYPROLINE WITH UNSATURATED FATTY ACIDS DERIVATIVES

(71) Applicant: MEDWELL LABORATORIES LTD., Nazareth (IL)

(72) Inventors: Taher NASSAR, Tur'an (IL); Michal OLSHANSKY, Tel Aviv (IL)

(21) Appl. No.: 14/894,001

(22) PCT Filed: May 28, 2014
(86) PCT No.: PCT/IL14/50481

§ 371 Date: Nov. 25, 2015

Related U.S. Application Data
(60) Provisional application No. 61/827,976, filed on May 28, 2013.
(60) Provisional application No. 61/827,986, filed on May 28, 2013.

(51) Int. Cl. A61K 31/4015; A61Q 19/08; A61K 8/49

(52) U.S. Cl. CPC A61K 31/4015; A61K 8/4913; A61Q 19/08

(57) ABSTRACT

Disclosed is a method for treating or preventing pathological or non-pathological skin conditions, disorders or diseases comprising administering a conjugate of proline, hydroxyproline, or a salt thereof, conjugated with a fatty acid.

### FIELD OF THE INVENTION

The invention is directed to methods of treating or preventing pathological and non-pathological skin conditions, disorders and diseases. The invention is further directed to the treatment of dry skin, aging skin, hyper-pigmentation, wrinkles as well as acne, irritant diaper rash, irritant dermatitis, psoriasis, rosacea, impetigo, burns and wounds.

### BACKGROUND OF THE INVENTION

The L form of proline (2-pyrrolidine-carboxylic acid) and 4-hydroxyproline (4-hydroxy 2-pyrrolidine-carboxylic acid, related to herein as hydroxyproline) are naturally occurring amino acids and are found, for example, in relatively large quantities in collagen. Elastins are also known to contain proline, in about each ninth residue. Proline and 4-hydroxyproline are not considered to be essential amino acids and further, no pharmacological activity for non-derivative proline and 4-hydrxyproline is known in the field.

However, certain N-substituted derivatives of proline or hydroxproline containing substituted peptides are known ACE-inhibitors and have become established for their ability to control high blood pressure (for example Captopril; Lisinopril; Enalapril and Moveltipril). Oxaceprol, an additionally N-acyl derivative; possesses antiphlogistic; anti-rheumatic and wound healing activity. Commercial infusions for parenteral nutrition occasionally contain proline as an adjuvant (for example, in combination with other amino acids, carbohydrates and electrolytes).

Certain Omega-3 and Omega-6 fatty acids are considered essential fatty acids, which, although essential for human health, cannot be produced by the human body. Other omega fatty acids are known to be conditionally essential, i.e., essential to the human body under certain conditions. Omega fatty acids can be found in fish, seafood and certain plants. Also known as polyunsaturated fatty acids (PUFAs), omega fatty acids play a crucial role in brain function, as well as normal growth and development. They have also become popular since they may reduce the risk of heart disease. Research shows that omega fatty acids reduce inflammation and may help lower risk of chronic diseases such as heart disease, cancer, and arthritis. Omega fatty acids are highly concentrated in the brain and appear to be important for cognitive and behavioral function. Symptoms of omega fatty acid deficiency include fatigue, poor memory, dry skin and heart problems.

Acne vulgaris is the most common disorder of human skin and is known to affects up to 80% of the population. Acne has many different symptoms including comedones, papules, pustules, nodules, cysts and pilosebaceous inflammation. Among these, inflammatory lesions of acne are of the greatest concern to patients because they may lead to acne scarring, thereby inducing adverse psychological effects. Propionibacterium acnes (referred to herein as P. acnes) is a Gram-positive anaerobic bacterium that mostly resides in the pilosebaceous follicles of the skin. Although P. acnes is a member of the normal skin commensal bacterial flora, it plays a critical role in the development of inflammatory acne when it overgrows and colonizes the pilosebaceous unit. It has also been widely accepted that inflammatory acne is induced by host immune reactions to P. acnes. P. acnes releases chemoactive factors that attract the immune system cells such as neutrophils, monocytes, and lymphocytes. Previous studies have found that P. acnes stimulates the production of proinflammatory cytokines such as interleukins-1b, -8, -12, and tumor necrosis factor-a. As reduction in P. acnes numbers in the hair follicle by antimicrobial agents correlates with clinical improvement of acne in patients, antibiotics have been used to treat acne for several decades and are still widely prescribed for acne patients.

Topical antimicrobial agents include benzoyl peroxide, clindamycin, erythromycin, tetracycline, azelaic acid, triclosan and various combinations of these agents. In cases where topical treatment is not successful or in patients at risk for scarring of the skin and pigmentary changes, systemic antibiotics are indicated. These include tetracycline, doxycycline, minocycline and erythromycin. However, data from several studies indicate a drastic increase in the proportion of patients carrying P. acnes strains resistant to one or more antibiotics. The relationship between skin colonization with resistant P. acnes isolates and treatment outcome is complex and the clinical significance of resistance is not always entirely clear. Nevertheless, careful analysis of clinical trials performed over the last two decades shows that there has been a gradual decrease in the efficacy of topical erythromycin, most likely related to the development of resistance. It is well established that bacterial biofilms play an important role in the pathogenesis of many human infections. One of the striking properties of sessile cells (i.e. cells growing in a biofilm) is their increased resistance to antimicrobial agents. Factors considered to be responsible for this phenotype include restricted penetration of antimicrobials, decreased growth rate, expression of resistance genes and the presence of resistant “persister” cells.

### SUMMARY OF THE INVENTION

Embodiments of the invention are directed to a method for treating or preventing pathological or non-pathological skin conditions, disorders or diseases comprising administering a conjugate of proline, hydroxyproline, or a salt thereof, conjugated with a fatty acid. According to some embodiments, the fatty acid is omega-3 fatty acid, omega-6 fatty acid or omega-9 fatty acid.

According to further embodiments, the conjugate is a compound of Formula (I)

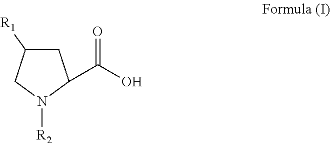

wherein R₁ is selected from hydrogen, unsaturated mono, poly and omega fatty acids, or OR₃;

R₂ and R₃ are independently selected from hydrogen or unsaturated mono, poly and omega fatty acids, provided that both R₁ and R₂ are not hydrogen and that if R₁ is OR₃, both R₂ and R₃ and not hydrogen.

According to further embodiments, the conjugate is a conjugate of hydroxyproline and docosahexaenoic acid (DHA) according to formula MW-001:

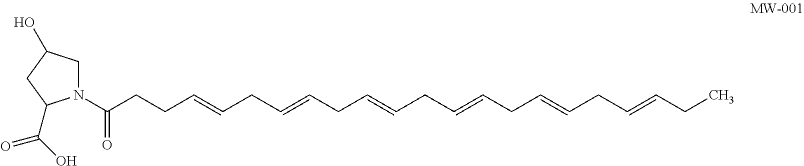 [SMILES: CC/C=C/C/C=C/C/C=C/C/C=C/C/C=C/C/C=C/CCC(=O)N1CC(O)CC1C(=O)O]

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

FIG. 1 is a bar graph presenting the change in the infected area before and after one and two weeks of treatment.

FIG. 2 is a bar graph presenting the change in the dryness level before and after one and two weeks of treatment.

FIG. 3 is a bar graph presenting the change in the scale level before and after one and two weeks of treatment.

FIG. 4 is a bar graph presenting the change in the redness level before and after one and two weeks of treatment.

FIG. 5 is a bar graph presenting the change in the lichenification level before and after one and two weeks of treatment.

FIG. 6 is a graph presenting the wrinkles appearance before, after two weeks and after four weeks of treatment.

FIG. 7 presents the volunteers assessment of wrinkles appearance after two and four weeks of treatment.

FIG. 8 presents the volunteers assessment of wrinkles condition around the eyes after two and four weeks of treatment.

FIG. 9 presents results of a questionnaire in which the volunteers were asked to their level of agreement with the statements detailed therein.

FIG. 10 presents a comparison between wrinkles around the eyes in the treated area and the control area.

FIG. 11 presents a comparison between the firmness of the skin in the treated area and the control area.

### DESCRIPTION OF THE DETAILED EMBODIMENTS OF THE INVENTION

According to one embodiment of the invention, there is provided a method for treating or preventing pathological and/or non-pathological skin conditions, disorders and diseases comprising administering an amino-acid or salts thereof, conjugated with any appropriate fatty acid or fatty acid derivative.

According to one embodiment of the invention, there is provided a method for treating or preventing pathological and non-pathological skin conditions, disorders and diseases comprising administering proline or hydroxyproline, or salts thereof, conjugated with any appropriate fatty acid or fatty acid derivative.

According to some embodiments, the conjugate is a compound of formula (I)

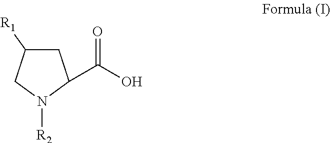

wherein R₁ is selected from hydrogen, unsaturated mono, poly and omega fatty acids, or OR₃;
R₂ and R₃ are independently selected from hydrogen or unsaturated mono, poly and omega fatty acids, provided that both R₁ and R₂ are not hydrogen and that if R₁ is OR₃, both R₂ and R₃ and not hydrogen. According to some embodiments, the omega fatty acid is omega-3 fatty acid, omega-6 fatty acid or omega-9 fatty acid.

According to some embodiments, the conjugate is a conjugate between hydroxyproline and an acid selected from group consisting of a lipoic acid, oleic acid, linoleic acid, arachidonic acid, eicosapentaenoic acid (EPA) and docosahexaenoic acid (DHA). According to some embodiments, the acid is DHA.

According to some embodiments, the conjugate is a conjugate of hydroxyproline and docosahexaenoic acid (DHA) according to formula MW-001:

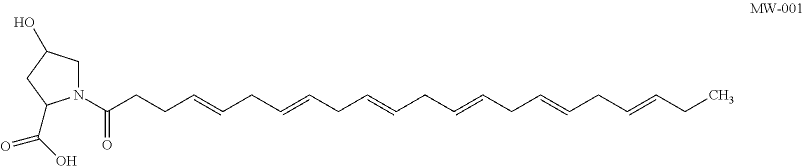 [SMILES: CC/C=C/C/C=C/C/C=C/C/C=C/C/C=C/C/C=C/CCC(=O)N1CC(O)CC1C(=O)O]

According to some embodiments of the invention, there is provided a method for inhibiting skin aging, including inhibiting or reducing skin wrinkles, sagging and/or dry skin comprising topically administering the conjugate of the invention or a topical, dermatological or cosmetic formulation including the conjugate of the invention. According to some embodiments, the conjugate of the invention or a topical, dermatological or cosmetic formulation including the conjugate of the invention improves skin aesthetics comprising topically administering the conjugate of the invention or a topical, dermatological or cosmetic formulation including the conjugate of the invention. According to further embodiments, the conjugate of the invention or a topical, dermatological or cosmetic formulation moisturizes the skin, thereby preventing and/or treating dry skin.

According to some embodiments, there is provided a method for reducing wrinkles comprising the step of administering a conjugate of proline, hydroxyproline, or a salt thereof, conjugated with a fatty acid.

According to some embodiments, there is provided a method for treating acne, irritant diaper rash, irritant dermatitis, psoriasis, rosacea, impetigo, forunculosis, burns and/or wounds, including chronic wounds comprising topically administering the conjugate of the invention or a topical, dermatological or cosmetic formulation including the conjugate of the invention. According to some embodiments, there is provided a method for treating acne vulgaris, acne variants, acne conglobata, acne mechanica, acne excoriee des jeunes filles, drug induced acne and/or occupational acne comprising topically administering the conjugate of the invention or a topical, dermatological or cosmetic formulation including the conjugate of the invention. According to some embodiments, there is provided a method for treating an infectious skin disease comprising topically administering the conjugate of the invention or a topical, dermatological or cosmetic formulation including the conjugate of the invention. According to some embodiments, there is provided a method for treating conditions caused by Propionibacterium acnes comprising topically administering the conjugate of the invention or a topical, dermatological or cosmetic formulation including the conjugate of the invention. According to further embodiments, there is provided a method for treating conditions caused by Staphylococcus spp comprising topically administering the conjugate of the invention or a topical, dermatological or cosmetic formulation including the conjugate of the invention.

According to some embodiments, the conjugate may be co-administered with any appropriate antibiotic. When administered to a patient together with an antibiotic, any type of co-administration is included herein. For example, the conjugate and the antibiotic may be administered in the same formulation, at the same time in different formulations or each having its own administration course, being possibly administered at different times to the same patient for treating the same condition. The antibiotic may be administered before, after or at the same time as the conjugate.

According to some embodiments, synergism is achieved when co-administering the conjugate and an antibiotic. According to some embodiments, synergism is achieved when co-administering a conjugate of hydroxyproline and DHA. According to some embodiments, such synergism allows the administration of relatively low doses of the conjugate and/or the antibiotic.

The conjugate may be administered by any appropriate means, including topically, orally or parenterally. According to some embodiments, the conjugate is administered topically in the form of a topical formulation.

According to some embodiments, the conjugate is administered together with any appropriate non-toxic pharmaceutical carrier. The carrier may be solid or liquid. According to some embodiments, the solid carrier is selected from lactose, corn starch, gelatin, talc, stearic acid, magnesium stearate, sucrose, agar, pectin, acacia or any combination thereof. The liquid carrier may be selected from peanut oil, olive oil, sesame oil, saline solution, water or any combination thereof.

According to some embodiments, the conjugate is administered together with any ingredients appropriate for modifying the release of the active conjugate from the formulation. Time delaying agents, such as glyceryl monostearate and glyceryl disearate may be used, with or without the addition of wax to the formulation.

According to some embodiments, the topical formulation further comprises one or more of an anti-inflammatory ingredient, anti-itch ingredient, anti-hystaminic ingredient, skin hydration ingredient, anti oxidant, anti microbial ingredient, skin barrier function enhancer, and any combination thereof. According to some embodiments, the topical formulation further comprises zinc. According to some embodiments, the topical formulation further comprises zinc oxide. According to further embodiments, the topical formulation further comprises silver ions. According to some embodiments, the topical formulation further comprises an anti fungal agent. According to some embodiments, the topical formulation further comprises hydrocortisone. According to some embodiments, the topical formulation further comprises a corticosteroid. According to some embodiments, the topical formulation further comprises a macrolide immunosuppressant. According to some embodiments, the topical formulation further comprises NSAIDS.

According to some embodiments of the invention, the pH of the topical formulation is between about 5.0-7.0. According to further embodiments of the invention, the pH of the topical formulation is between about 5-5.5. According to further embodiments of the invention, the pH of the topical formulation is between about 5.5-6.0. According to further embodiments of the invention, the pH of the topical formulation is between about 6.0-6.5. According to further embodiments, the pH of the topical formulation is between about 6.5-7.0. According to further embodiments of the invention, the pH of the topical formulation is between about 5.0-6.5.

The topical skin formulation may be in the form of a gel, an ointment, pomade, a body and/or face soap, a cream, a cleansing gel, a lotion, oil-in-water, water-in-oil, water-in-oil-in-water, and oil-in-water-in-silicone emulsions, an aqueous solution, a paste, a foam, a salve, an oil, a wash, a conditioner or an aerosol. The formulation may be applied in any suitable manner, e.g., by hand, spatula, spray or pad.

The topical formulation may further comprise cosmetic or dermatological acceptable ingredients or dermatologically acceptable carriers. According to some embodiments of the invention, the compositions may include one or more of an oil, a fat, wax, a detergent, a conditioner, a pH modifier, a preservative, a solvent, a viscosity modifier, a colorant, a perfume, a dyestuff and the like.

According to an embodiment of the invention, the compositions of the invention are administered once a day. According to other embodiments, the compositions are administered twice a day, three times a day or more.

According to an embodiment of the invention, the composition is administered chronically. According to some embodiments of the invention, the composition is administered for about 10 days or more, 20 days, 30 days, 60 days, 90, 120 days or more.

In some embodiments of the invention, the composition further comprises a cleaning agent or a detergent that are typically anionic, cationic, non-ionic or amphoteric surfactants. Typical anionic surfactants are carboxylates, sulfonates, sulfates or phosphates, e.g. fatty acid soaps, salts of lauryl sulfate and salts of lauryl ether sulfate. Examples of cationic surfactants are aliphatic mono, di and polyamines derived from fatty and rosin acids, amine oxides, ethoxylated alkyl amines and imidazolines. Examples of non-ionic surfactants are polyoxyethylene surfactants, alkylphenol ethoxylates, carboxylic acid esters, e.g., mono and diglycerides, polyoxyethylene esters and fatty acid diethanolamine condensates. Amphoteric surfactants are those containing combinations of the anionic and cationic groups described above, particularly those containing both acid carboxyls and basic nitrogen groups. Typical amphoteric surfactants are imidazolines and betaines, e.g., lauric and myristic imidazolines and betaines, and amidopropylbetaines.

The topical pharmaceutical compositions may also comprise a suitable emulsifier, which refers to an agent that enhances or facilitates mixing and suspending oil-in-water or water-in-oil. The emulsifying agent used herein may consist of a single emulsifying agent or may be a nonionic, anionic, cationic or amphoteric surfactant or blend of two or more such surfactants. Such surface-active agents are described in “McCutcheon's Detergent and Emulsifiers,” North American Edition, 1980 Annual published by the McCutcheon Division, MC Publishing Company, 175 Rock Road, Glen Rock, N.J. 07452, USA.

The CTFA Cosmetic Ingredient Handbook, Second Edition (1992) describes a wide variety of non-limiting cosmetic ingredients commonly used in the skin care industry, which are suitable for use in the topical formulation. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizate), skin treating agents, thickeners, and vitamins and derivatives thereof.

According to some embodiments, the conjugate is administered, together with any additional appropriate ingredients as a formulation in the form of a food, feed, food additive, feed additive, drink or drink additive. According to some embodiments, the formulation is in the form of tablets, capsules, powders, troches, soft gelatin capsules, syrup, liquid suspension or lozenges and may be administered orally. According to some embodiments, the conjugate is prepared into a parenteral formulation, such as for subcutaneous intramuscular or intravenous administration.

As defined herein, treating and preventing includes fully healing a condition, partially healing a condition, such that an improvement occurs and preventing, or at least partially preventing, the occurrence of the condition.

The term “preventing” refers to keeping a disease, disorder or condition from occurring in a subject. In some cases the subject may be at risk for developing the disease, but has not yet been diagnosed as having the disease. In some instances, the term “preventing” refers to preventing the next cycle of the disease from occurring.

### EXAMPLES

In Examples 1-3 an emulsion type O/W Creams of MWL-001 (2.5% and 5% w/w) was prepared using Emulgade® SE emulsifiers (glyceryl stearate), Ceteareth-20, cetearyl alcohol, cetyl palmitate, cetearyl alcohol-Lanette®, isopropyl myristate, caprilic/capric acid triglycerides (Myritol®318), strearic acid, a Trietanolamine and purified water, according to Ph Eur 6.0.

### Example 1

### The Efficacy of MW-001 in the Treatment of Atopic Dermatitis

### Introduction

Atopic dermatitis is a pruritic disease of unknown origin that usually starts in early infancy and is typified by pruritus, eczematous lesions, xerosis (dry skin), and lichenification of the skin (thickening of the skin and an increase in skin markings). The disease usually appears on the face, on the inner side of the elbows, behind the knees, and on the hands and feet. Atopic dermatitis is associated with other atopic diseases (eg, asthma, allergic rhinitis, urticaria, acute allergic reactions to foods and increased immunoglobulin E ([IgE] production) in many patients. It is a disease with a high rate of morbidity, and its occurrence appears to be on the increase.

### Inclusion Criteria

Volunteers aged 2-30 years, with Atopic dermatitis.

### Exclusion Criteria

Volunteers with a known allergy to one of the tested materials or to any of the ingredients.

Treatment with medication such as anti-inflammatories, anti-histamines, corticosteroids, systemically or topically applied, unless stopped 2 weeks prior to the trial in the case of systemic treatment and 3 days in the case of topical treatment.

Volunteers in the process of diagnosis or treatment for cancer, kidney disease or liver disease.

Pregnant or lactating women.

### Test Performance

Ten volunteers aged 2-30 years participated in the trial. Four females and six males.

Volunteers were instructed to apply the product only on one side of the body, on the worst affected area, while the other side was defined as the control area and was not treated. The tested material, a body lotion product, was applied 1-3 times a day for a period of 4 weeks.

The volunteers were instructed to avoid using any other treatment during the test period.

Each volunteer was examined at baseline (T0), after one week (T1) and after 2 weeks of using the product (T2). The investigator rated the condition of the skin according to the parameters of the size of the affected area, dryness, scale, redness, edema, lichenification and pruritus. Both the treated and the Control areas were documented with color photographs at T0, T1 and T2.

The participants were asked to keep a record of the daily applications in a diary and to note any side effects resulting from the product application.

### Examination Time-Points

At baseline (T0)

After one week of treatment (T1)

After two weeks of treatment (T2)

### Measurements

### Hydration Level

Hydration level was evaluated by Comeometer CM 825, Courage & Khazaka, CK. The probe of the Corneometer was placed on the skin at a constant pressure and measurements of skin hydration were taken. Skin hydration was recorded at baseline, after 1 week and after 2 weeks of treatment. Room temperature and humidity were kept constant.

### Erythema Level

Erythema level was evaluated by Mexameter, Courage & Khazaka, CK. The probe of the Mexameter was placed on the skin at a constant pressure and measurements of skin hydration were taken. Skin erythema level was recorded at baseline, after 1 week and after 2 weeks of treatment.

### Trans Epidermal Water Loss

Transepidermal water loss (TEWL) was measured by Tewameter 300 TM, Courage & Khazaka, CK. TEWL was recorded at baseline and after one week and after two weeks of treatment. Room temperature and humidity were kept constant.

### Study Director's Evaluation

The following parameters were evaluated:
a. Lesion area
b. Dryness
c. Scales
d. Redness
e. Edema
f. Lichenificalion

The parameters mentioned above were evaluated on a scale of 0 to 5 by the study director.
0=None
1=Slight
2=Moderate
3=Severe
4=Extreme
5=Very Extreme

### Subjective Evaluation

The volunteers were asked to assess the pruritus intensity on a 0-5 scale at T0, T1 and T2.
0=None
1=Slight
2=Moderate
3=Severe
4=Extreme
5=Very Extreme

### Side Effects

In addition to keeping records of side effects, volunteers were asked to immediately report any side effects, such as dryness, burning sensation, peeling of the skin, redness, etc. to 1SR.

### Statistical Analysis

A statistical comparison between T0 and the other two time-points (T1 and T2) and between the treated and the Control areas were made according to the Wilcoxon signed-ranks test, while p<0.05 indicated a significant difference

### Test Results

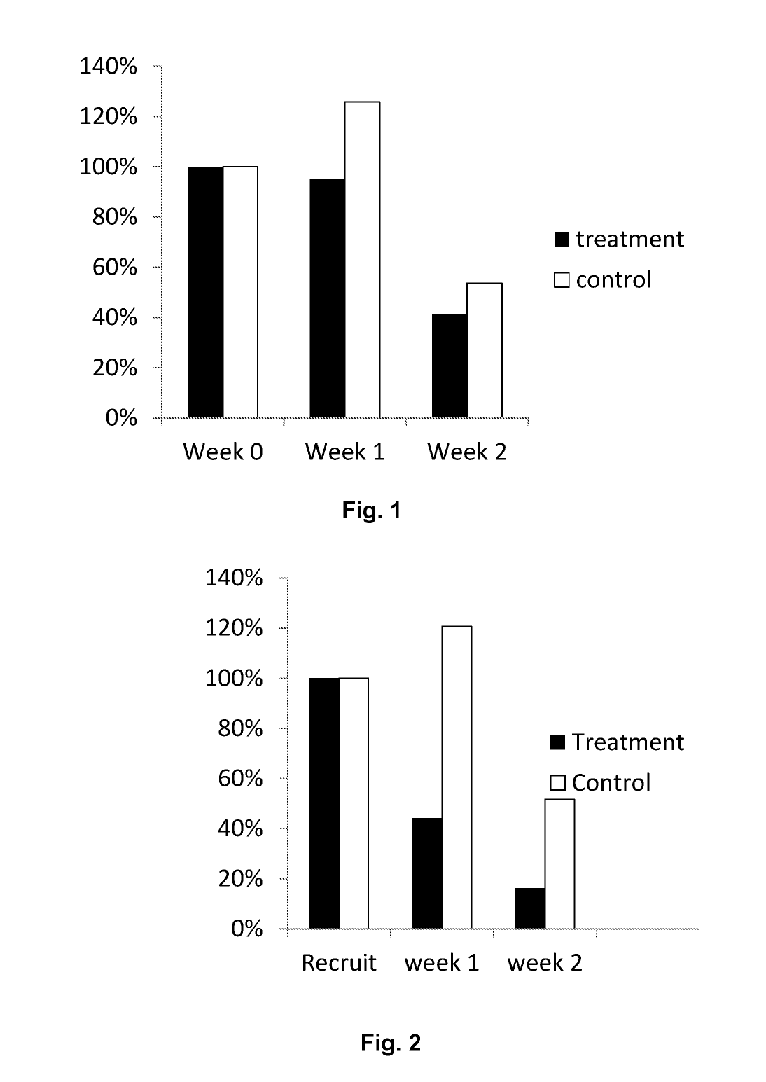

The results of 10 volunteers at each time point are presented in FIGS. 1-5. All parameters were assessed in a scale of 0-5 where 0=none and 5=very extreme. The percentage of change was calculated at T1 and T2.

TABLE I
Infected area size

| | T0 | T1 | T2 |
|---|---|---|---|
| Treatment | 4.3 | 3.2 | 2.2 |
| Control | 3.3 | 3.9 | 2.8 |

Treated area: At T0, the infected area size assessment was 4.3. At T1 the infected area size assessment was 3.2. At T2 the infected area size was 2.2.

Control area: At T0, the infected area size assessment was 3.3. At T1 the infected area size assessment was 3.9. At T2 the infected area size was 2.8.

### Statistical Analysis

The infected area size at the treated area diminished significantly after 2 weeks of treatment (p<0.05). Results showing the change in the infected area are presented in FIG. 1.

TABLE II
Dryness level

| | T0 | T1 | T2 |
|---|---|---|---|
| Treatment | 4.1 | 1.8 | 1.1 |
| Control | 3.1 | 3.2 | 1.8 |

Treated area: At T0, the dryness level was 4.1. At T1 the dryness level was 1.8. At T2 the dryness level was 1.1.

Control area: At T0, the dryness level was 3.1. At T1 the dryness level was 3.2. At T2 the dryness level was 1.8.

### Statistical Analysis

After 1 week of treatment, the dryness level decreased significantly at the treated area. After 2 weeks of treatment, the dryness level of both sides decreased significantly. Results showing the change in the dryness level are presented in FIG. 2.

TABLE III
Scale level

| | T0 | T1 | T2 |
|---|---|---|---|
| Treatment | 2.6 | 1.3 | 0.9 |
| Control | 2.2 | 2.2 | 1.5 |

Treated area: At T0, the scale level was 2.6. At T1 the scale level assessment was 1.3. At T2 the scale level was 0.9.

Control area: At T0, the scale level was 2.2. At T1 the scale level stayed 2.2. At T2 the scale level was 1.5.

### Statistical Analysis

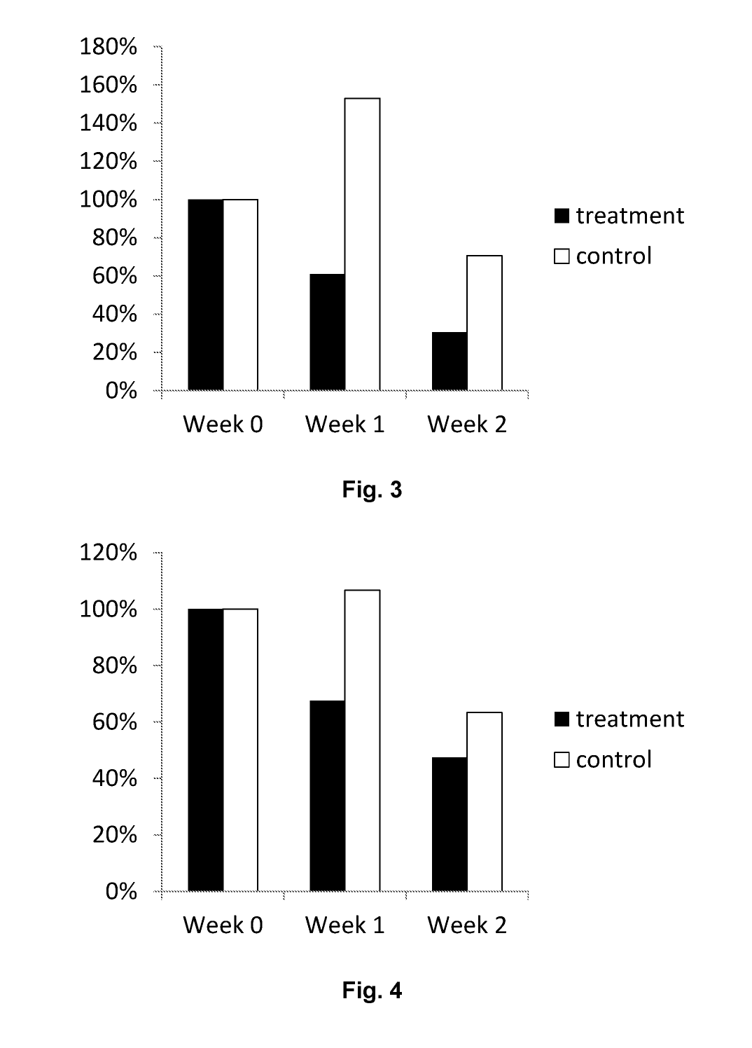

After 1 and 2 weeks of treatment, the scale level decreased significantly only at the treated area. Results showing the change in the scale level are presented in FIG. 3.

TABLE IV
Redness level

| | T0 | T1 | T2 |
|---|---|---|---|
| Treatment | 3.9 | 2.4 | 2.3 |
| Control | 3.1 | 3.1 | 2.1 |

Treated area: At T0, the redness level was 3.9. At T1, the redness level was 2.4. At T2 the redness level was 2.3.

Control area: At T0, the redness level was 3.1. At T1, the redness level was 3.1. At T2 the redness level was 2.1.

### Statistical Analysis

After 1 week of treatment, the redness level decreased significantly at the treated area. After 2 weeks of treatment, the redness level of both sides decreased significantly. Results showing the change in the redness level are presented in FIG. 4.

TABLE V
Lichenification level

| | T0 | T1 | T2 |
|---|---|---|---|
| Treatment | 2.8 | 2.0 | 1.3 |
| Control | 2.1 | 2.7 | 1.7 |

Treated area: At T0, the lichenification level was 2.8. At T1, the lichenification level was 2.0. At T2 the lichenification level was 1.3.

Control area: At T0, the lichenification level was 2.1. At T1, the lichenification level was 2.7. At T2 the lichenification level was 1.7.

### Statistical Analysis

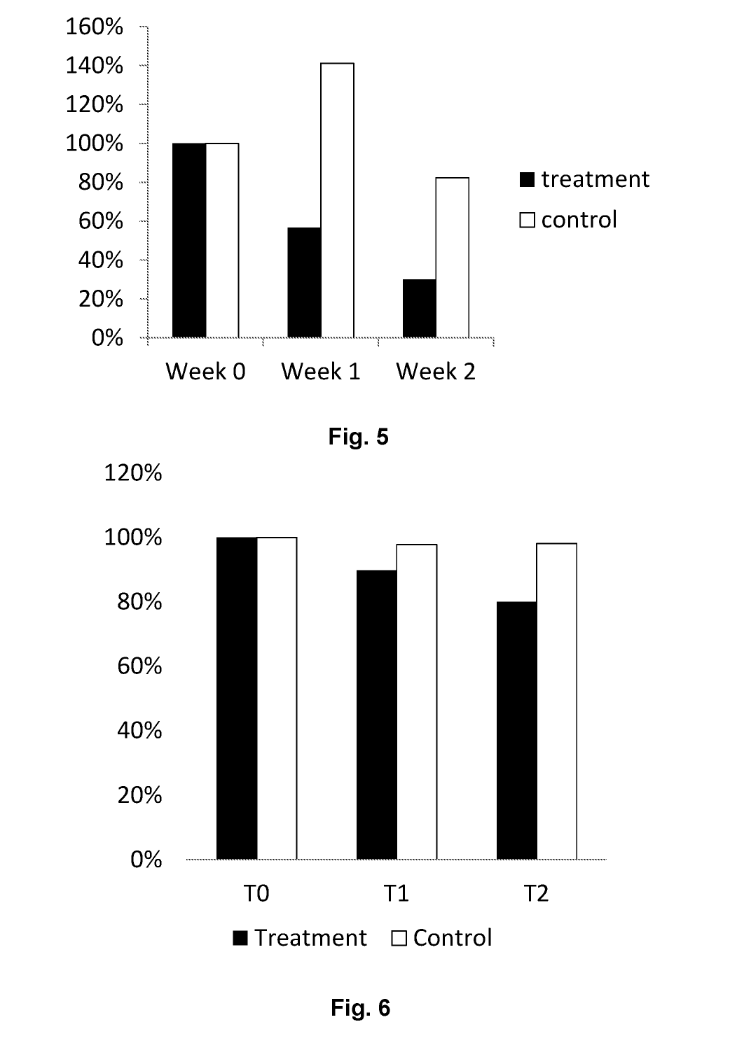

After 2 weeks of treatment, the lichenification level decreased significantly only at the treated area. Results showing the change in the lichenification level are presented in FIG. 5.

TABLE VI
Pruritus level

| | T0 | T1 | T2 |
|---|---|---|---|
| Treatment | 3.4 | 1.4 | 0.9 |
| Control | 2.7 | 2.6 | 2.0 |

TABLE VII
Change in Pruritus level

| | Baseline | Week 1 | Week 2 |
|---|---|---|---|
| Treatment | 100% | 41% | 26% |
| Control | 100% | 99% | 75% |

Treated area: At T0, the pruritus level was 3.4. At T1 the pruritus level was 1.4. At T2 the pruritus level was 0.9.

Control area: At T0, the pruritus level was 2.7. At T1 the pruritus level was 2.6. At T2 the pruritus level was 2.0.

### Statistical Analysis

After 1 and 2 weeks of treatment, the pruritus level decreased significantly only at the treated area.

TABLE VIII
Hydration level

| | T0 | T1 | T2 |
|---|---|---|---|
| Treatment | 21.1 | 30.7 | 27.8 |
| Control | 23.9 | 26.2 | 33.9 |

TABLE IX
Change in hydration level

| | Baseline | Week 1 | Week 2 |
|---|---|---|---|
| Treatment | 100% | 146% | 132% |
| Control | 100% | 110% | 142% |

Treated area: The hydration level at the treated area was 21.1 at T0, increased to 30.7 at T1 and increased to 27.8 at T2.

Control area: The hydration level at the Control area was 23.9 at T0, 26.2 at T1 and 33.9 at T2.

### Statistical Analysis

There was no significant change in hydration level at any of the sides

TABLE X
TEWL level

| | T0 | T1 | T2 |
|---|---|---|---|
| Treatment | 13.6 | 12.5 | 10.5 |
| Control | 9.6 | 16.7 | 15.9 |

TABLE XI
Change in TEWL level

| | Baseline | Week 1 | Week 2 |
|---|---|---|---|
| Treatment | 100% | 92% | 78% |
| Control | 100% | 174% | 165% |

Treated area: The TEWL level at the treated area was 13.6 at T0, decreased to 12.5 at T1 and decreased to 10.5 at T2.

Control area: The TEWL level at the Control area was 9.6 at T0, increased to 16.7 at T1 and increased to 15.9 at T2.

### Statistical Analysis

After 2 weeks of treatment, the TEWL level at the control area increased significantly.

TABLE XII
Erythema level

| | T0 | T1 | T2 |
|---|---|---|---|
| Treatment | 620.5 | 562.7 | 600.2 |
| Control | 604.5 | 604.8 | 584.6 |

TABLE XIII
Change in erythema level

| | Baseline | Week 1 | Week 2 |
|---|---|---|---|
| Treatment | 100% | 91% | 97% |
| Control | 100% | 100% | 97% |

Treated area: The erythema level at the treated area was 620.5 at T0. At T1 the erythema level was 562.7 level and at T2 the erythema level was 600.2.

Control area: The erythema level at the control area was 604.5 at T0, 604.8 at T1, and 600.2 at T2.

### Statistical Analysis

After 1 week of treatment, the erythema level decreased significantly at the treated side, but then increased again and was not significantly different from the erythema level at baseline.

Referring to the use of the cream, volunteers were asked to fill out a questionnaire expressing their level of agreement with the statements listed in the questionnaire on a Scale of 1-10, where the worst score was 1 and an excellent score was 10. The volunteers assessed the product as follows: long lasting moisture-7.4; pleasantness-8.6; and total score was 8.7.

### Drop-Outs

None of the participants dropped out from the test.

### Side Effects

None of the participants had side effects due to product use

### Summary

After two weeks of treatment with the product MW-001 (related to herein also as MWL-001) there was a significant improvement in the infected area size, dryness, redness, scale, lichenification and pruritus at the treated side. There was also a significant improvement in redness and dryness parameters at the control side.

All the volunteers were very satisfied with the product, and have very high intentions of purchasing the product in the future.

The product MW-001 was found to be safe for use and very efficient in the treatment of atopic dermatitis.

### Example 2

### The Efficacy of MW-001 in the Treatment of Psoriasis

### Introduction

Psoriasis is a chronic skin disease common in 2-3% of the population characterized by faster and uncontrolled growth of skin cells in specific areas as much as 27 times more than in healthy skin cells. As a result, the skin cells accumulate layer on layer thus creating thick skin with “plaque” and inflamed areas are covered with silvery thick white scales. The affected areas in Psoriasis Vulgaris are usually the elbows, knees, scalp, back and finger nails

### Inclusion Criteria

Volunteers aged 18-65 with psoriasis

The study director assigned a serial number to each volunteer.

Volunteers signed an informed consent form prior to the test and after detailed explanation.

### Exclusion Criteria

Volunteers with a known allergy to one of the tested materials or their ingredients.

Treatment with medication such as anti-inflammatories, antihistamines, corticosteroids, systemically or topically applied, unless stopped 2 weeks prior to the trial in the case of systemic treatment and 3 days before in the case of topical treatment.

Pregnant or lactating women.

### Methodology

Fifteen volunteers aged 29-73 with psoriasis.

The volunteers signed a consent form prior to the test and after detailed explanation.

The volunteers were instructed to use the cream at least once a day, for 4 weeks.

Volunteers were instructed to apply the product only on one side of the body, on the worst affected area at least once a day, for 4 weeks, while the other side was defined as the control area and was not treated.

The volunteers were instructed to avoid using any other treatment during the test period.

Each volunteer was examined at baseline (T0), after two weeks (T1) and after 4 weeks of using the product (T2). The study Director rated the state of the skin according to the parameters of scales severity, thickness of the plaque, redness and the size of the lesions. Erythema level was documented at each time point.

Each volunteer filled out a questionnaire regarding the tested cream at the end of the trial.

The participants were asked to keep a record of daily applications and to note any side effects resulting from product application.

### Examination Time-Points

At baseline (T0)

After two weeks of treatment (T1)

After four weeks of treatment (T2)

### Measurements

### Erythema Level

Erythema level was evaluated by Mexameter, Courage & Khazaka, CK. The probe of the Mexameter was placed on the skin at a constant pressure find measurements of skin hydration were taken. Skin erythema level was recorded at baseline, after 2 weeks and after 4 weeks of treatment.

### Study Director's Evaluation

The following parameters were evaluated:

a) Size of lesion
b) Thickness of lesion

### c) Redness

### d) Scales

The parameters mentioned above were evaluated on a scale of 0 to 5 by the study director.

### 0=Absent

### 1=Slight

### 2=Moderate

### 3—Severe

### 4=Extreme

### 5—Very Extreme

### Subjective Evaluation

The volunteers were asked to assess the pruritus level on a 0-5 scale at T0, T1 and T2.

### 0=Absent

### 1=Slight

### 2=Moderate

### 3=Severe

### 4=Extreme

### 5=Very Extreme

### Side Effects

Volunteers were asked to keep a record and report any side effects, such as dryness, burning sensation, peeling of the skin, redness or any other visible changes, and report immediately to study director.

### Statistical Analysis

A statistical comparison between T0 and the other two time-points (T1 and T2) and between the treated and the Control areas were made by paired sample t-test, while p<0.05 indicated a significant difference

### Test Results

The results of 15 volunteers at each time point are presented in the Tables below and in the relevant Figures. All parameters were assessed in a scale of 0-5 where 0=none and 5=very extreme

### Infected Area Size

TABLE XVI

| | T0 | T1 | T2 |
|---|---|---|---|
| Treatment | 4.1 | 3.8 | 3.9 |
| Control | 3.4 | 3.5 | 3.3 |

### Treated Area:

At T0 the infected area size assessment was 4.1. At T1 the infected area size assessment was 3.8. At T2 the infected area size was 3.9.

### Control Area:

At T0 the infected area size assessment was 3.4. At T1 the infected area size assessment was 3.5. At T2 the infected area size was 3.3.

### Statistical Analysis

A significant decrease in lesion area size was observed in the treatment area after 2 weeks of application.

### Thickness of the Skin

TABLE XV

| | T0 | T1 | T2 |
|---|---|---|---|
| Treatment | 3.0 | 2.6 | 2.4 |
| Control | 2.9 | 3.3 | 3.0 |

### Treated Area:

At T0 the thickness level was 3.0. At T1 the thickness level was 2.6. At T2 the infected area size was 2.4.

### Control Area:

At T0 the thickness level was 2.9. At T1 the thickness level was 3.3. At T2 the infected area size was 3.0.

### Statistical Analysis

A significant improvement in thickness parameter was observed in the treatment area after 4 weeks (p<0.05).

### Redness Level

TABLE XVI

| | T0 | T1 | T2 |
|---|---|---|---|
| Treatment | 3.3 | 3.5 | 3.2 |
| Control | 3.3 | 3.1 | 3.4 |

### Treated Area:

At T0 the redness level was 3.3. At T1 the redness level was 3.5. At T2 the redness level was 3.2.

### Control Area:

At T0 the redness level was 3.3. At T1 the redness level was 3.1. At T2 the redness level was 3.4.

### Statistical Analysis

No significant change in redness parameter was observed in any of the sides during the whole study period (p>0.05).

### Scale Level

TABLE XVII

| | T0 | T1 | T2 |
|---|---|---|---|
| Treatment | 4.5 | 4.1 | 3.8 |
| Control | 4.4 | 4.6 | 4.8 |

Treated area: At T0 the scales level was 4.5. At T1 the scale level, assessment was 4.1. At T2 the scale level was 3.8.

Control area: At T0 the scales level was 4.4. At T1 the scale level was 4.6. At T2 the scale level was 4.8.

### Statistical Analysis

A significant improvement in scales parameter was observed in the treatment area after 4 weeks (p<0.05)

### Pruritus Level

TABLE XVIII

| | T0 | T1 | T2 |
|---|---|---|---|
| Treatment | 2.1 | 1.6 | 1.3 |
| Control | 1.9 | 1.6 | 1.9 |

Treated area: At T0 the pruritus level was 2.1. At T1, the pruritus level was 1.6. At T2 the pruritus level was 1.3.

Control area: At T0 the pruritus level was 1.9. At T1 the pruritus level was 1.6. At T2 the pruritus level was 1.9.

### Statistical Analysis

No significant change in pruritus level was observed in any of the sides during the whole study period (p>0.05)

### Erythema Level

TABLE XIX

| | T0 | T1 | T2 |
|---|---|---|---|
| Treatment | 562.1 | 517.5 | 576.7 |
| Control | 537.3 | 478.4 | 529.1 |

Treated area: The erythema level at the treated area was 562.1 at T0, 517.5 at T1 and 576.7 at T2.

Control area: The erythema level at the control area was 537.3 at T0, 478.4 at T1, and 529.1 at T2.

### Statistical Analysis

After 2 weeks of treatment, a significant decrease in erythema level was observed in the control side (p<0.05)

Volunteers were asked to answer a questionnaire relating to the use of the cream and to assess the different qualities of the product and grade the product in a scale of 1-10, where 1 was the worst score, and 10 was the best score. The volunteers assessed the product as follows: ease of application-9.1; scent-8.4; spreading on the skin-8.2; long lasting hydration-5.6; pleasant sensation-6.7; texture-6.5; and total score was 7.2.

### Side Effects

No side effects were observed in this study

### Summary

The general score given to this product was 7.2 in a scale of 1-10.

The highest scores were for the parameters of scent (8.4) and ease of application (9.1)

A significant improvement in the parameters of scales and thickness of the lesion were observed after 4 weeks of application only at the treated side.

After 2 weeks of application, there was a significant decrease in the lesion area size, but after 4 weeks of application, the decrease was found to be insignificant.

Significant differences between the treatment side and the control side in scale and thickness parameters were observed after both 2 weeks (T1) and 4 weeks (T2) of application.

Eight out of 14 volunteers answered that the previous product in use was better than the study product. Four of these volunteers used steroids and the other 4 used natural products.

The results of the study indicate that the product MW-001 is efficient in the treatment of psoriasis and safe for use.

### Example 3

### Efficacy, Safety and Tolerance Evaluation Test of the MW-001

### Objectives

Evaluation of the anti wrinkle effect of the products MWL-001 after 4 weeks of use by means of photography, objective assessment, and a questionnaire as well as silicon replica measuring the skin roughness, and an evaluation of the effect on skin hydration (moisture) and TEWL (Trans Epidermal Water Loss).

Number of Subjects: 10

### Inclusion Criteria

Female subjects aged 40 years and over.

Absence of any visible skin disease that might be confused with a skin reaction to the test material.

No known allergies to skin care products.

Completion of a Medical history form and the understanding and signing of an Informed Consent form.

Considered reliable and capable of following directions.

### Exclusion Criteria

Volunteers with a known allergy to one of the tested materials or to any of the ingredients.

Treatment with medication such as anti-inflammatories, anti-histamines, corticosteroids, systemically or topically applied, unless stopped 2 weeks prior to the trial in the case of systemic treatment and 3 days in the case of topical treatment.

Volunteers in the process of diagnosis or treatment for cancer, kidney disease or liver disease

Pregnant or lactating women.

### Study Design

Ten healthy female volunteers were included and completed the study.

Prior to the study, each of the subjects received a detailed explanation from the investigator and signed an informed consent.

A subjective assessment of facial wrinkles condition was performed by the volunteers at each time point.

Color photography was taken at each time point.

Moisture level of the skin (corneometry) and TEWL (Trans Epidermal Water Loss) were measured at each time point.

Silicon imprints were taken from the crow's feet area around the eyes at both treated and control sides at each time point.

A cosmetic anti wrinkle product MW-001 was applied on half of the face for four weeks by each volunteer while the other half served as a control, without any product application during the whole test period.

After 2 and 4 weeks of the same parameters of corneometry, TEWL, color photography, silicone imprints and subjective assessment by the volunteers of wrinkles condition of both treated and control sides.

The volunteers filled out a questionnaire after 2 and 4 weeks of application.

### Examination Time-Points

a. At baseline (T0)
b. After 2 weeks of use (T1)
c. After 4 weeks of use (T2)

### Methodology

### Measurements

### Hydration Level

Hydration level was evaluated by Corneometer, Courage & Khazaka, CK. The probe of the corneometer was placed on the skin at a constant pressure and measurements of skin hydration were taken. Skin hydration was recorded at T0, T1 and T2. Each measurement was repeated three times, and the average of the repeated measurements was calculated and assembled in the test results. The room temperature and humidity were kept constant.

### TEWL

TEWL (the ability not to harm the skin barrier) was measured with Tewameter TM 300, Courage & Khazaka, CK. TEWL was recorded at T0, T1 and T2. The results of TEWL in each measurement were recorded after stabilization of the curve of the results in real time. Room temperature and humidity were constant.

### Silicone Imprints Assessment

A computer program draws a realistic density three-dimensional image of the relief of the skin on a colour screen for a particular density of points selected for x and y axes and then the measurements data stored in the computer are processed and then evaluated.

This analysis consists of the following steps:

1. Generation of a roughness profile from the surface relief by alignment and filtering.
2. Calculation of standardized roughness parameters DIN 4768 ff). The silicone imprints were taken only at the treated side.

### Photography

Color photography of the treated area was taken at T0 (baseline), T1 (after 2 weeks of treatment) and T2 (after 4 weeks of treatment), in order to record the wrinkles' appearance.

### Subjective Evaluation (Questionnaire)

The volunteers were asked to answer a questionnaire after four weeks of application, regarding the assessment of each of the tested products, and to evaluate the improvement of the skin beneath and at the side of the eye.

### Side Effects/Adverse Events

Volunteers were asked to report side effects, such as dryness, burning sensation, peeling of the skin, redness, etc. immediately to the study director.

### Statistical Analysis

A statistical comparison between the results at T0 and at T1 was made according to the Wilcoxon signed-ranks test, a very sensitive paired-samples non-parametric test for small groups with unknown distribution, where p<0.05 indicates a significant difference. The software used was SPSS 14.

### Corneometry for Moisture Measurements

The corneometry results are provided in arbitrary units.

The average hydration level in the treated area was 60.9 at T0, 53.4 at T1 and 53.5 at T2.

The average hydration level in the control area was 61.7 at T0, 57.6 at T1 and 50.8 at T2.

### TEWL—Statistical Analysis

The average TEWL level in the treated area was 7.8 g/hm² at T0, 9.4 g/hm² at T1 and 7.6 g/hm² at T2.

The average TEWL level in the Control area was 6.2 g/hm² at T0, 8.8 g/hm² at T1 and 5.9 g/hm² at T2.

Wrinkles Appearance with Silicone Imprints

A graph presenting results of wrinkles appearance with silicone imprints is presented in FIG. 6.

Wrinkle appearance by silicone imprints analysis at the treated area:
Wrinkle's appearance with silicone imprints of the treated area, compared before and after 2 weeks of use, showed the following results: Decrease of skin roughness by 10.8% at the treated area.
Wrinkle's appearance with silicone imprints of the treated area, compared before and after 4 weeks of use, showed the following results: Decrease of skin roughness by 19.5% at the treated area.
The improvement at both T1 and T2 was significant (p<0.05)

Wrinkle appearance by silicone Imprints analysis at the control area:
Wrinkle's appearance with silicone imprints of the control area, compared before and after 2 weeks of use, showed the following results: Decrease of skin roughness by 0.9% at the Control area.
Wrinkle's appearance with silicone imprints of the control area, compared before and after 4 weeks of use, showed the following results: Decrease of skin roughness by 1.2% at the Control area.
The improvement at both T1 and T2 was found to be not significant (p>0.05)

### Questionnaire Results:

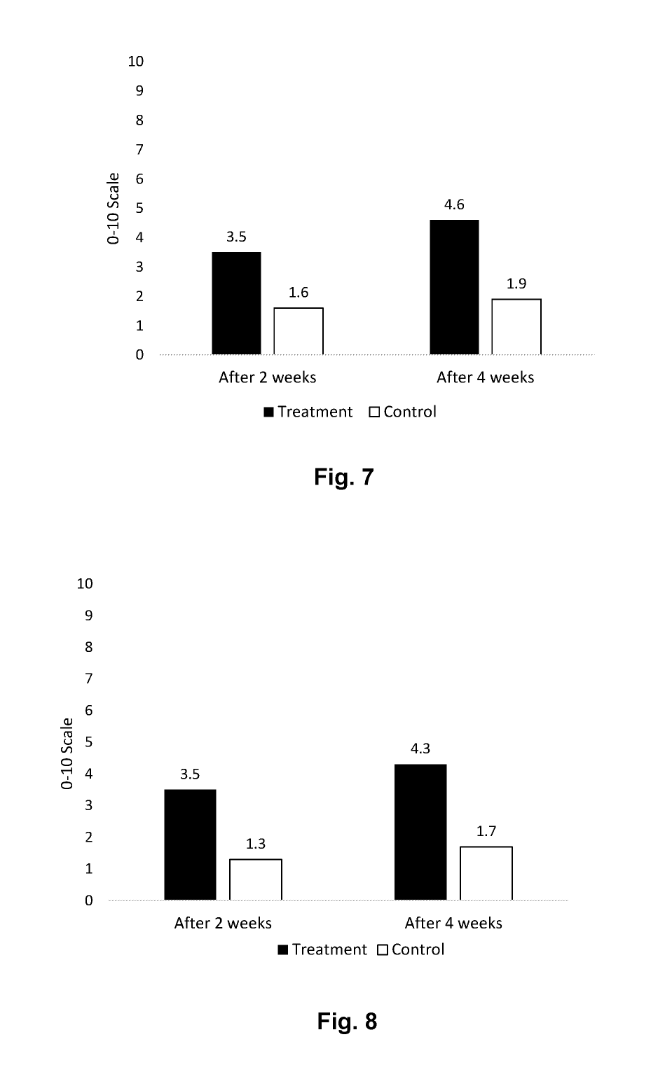

The volunteers were asked to assess the improvement in wrinkles appearance of both the treated and the Control sides at T1 and T2 in a scale of 0-10, where 0=no change and 10=very significant improvement. The results are presented in FIG. 7.

After 2 weeks of treatment: The volunteers rated the improvement of wrinkle appearance at the treated side with a grading of 3.5, and the control area with a grading of 1.6.

After 2 weeks of treatment: The volunteers rated the improvement in wrinkles appearance at the treated side with a grading of 4.6, and the control area with a grading of 1.9.

The volunteers were asked to assess the improvement of wrinkle condition around the eyes at both the treated and the control areas at T1 and T2 in a scale of 0-10, where 0=no change and 10=very significant improvement. The results are presented in FIG. 8.

After 2 weeks of treatment: The volunteers rated wrinkle improvement at the treated side with a grading of 3.5 and the control area with a grading of 1.3.

After 4 weeks of treatment: The volunteers rated wrinkle improvement at the treated side with a grading of 4.3, and the control area with a grading of 1.7.

FIG. 9 presents results of a questionnaire in which the volunteers were asked to their level of agreement with the statements appearing on the questionnaire, wherein the scale was from 1-4 (1=total disagreement, 4=total agreement).

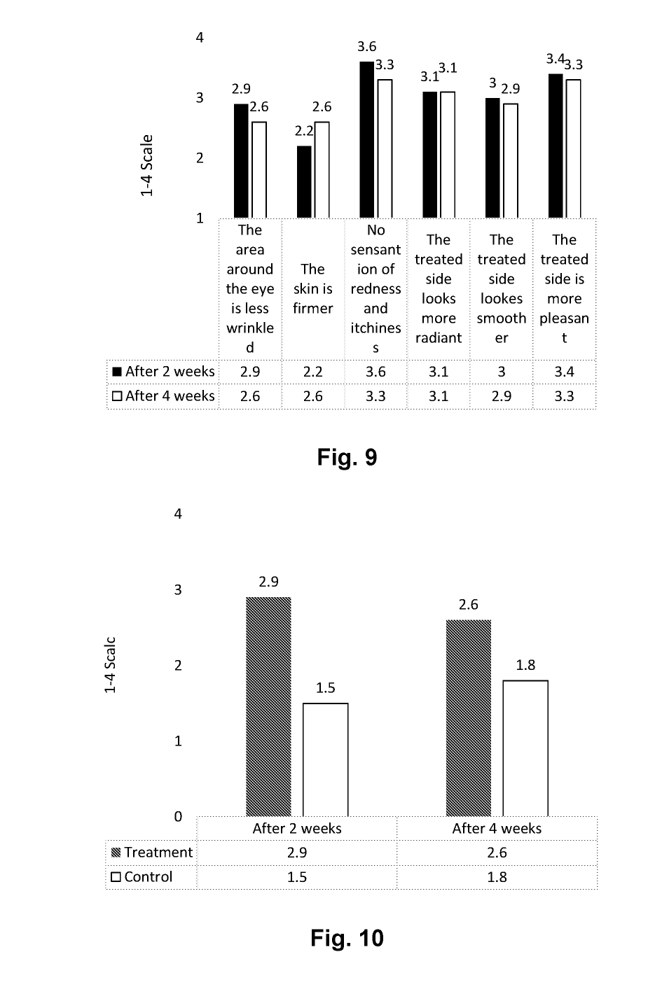

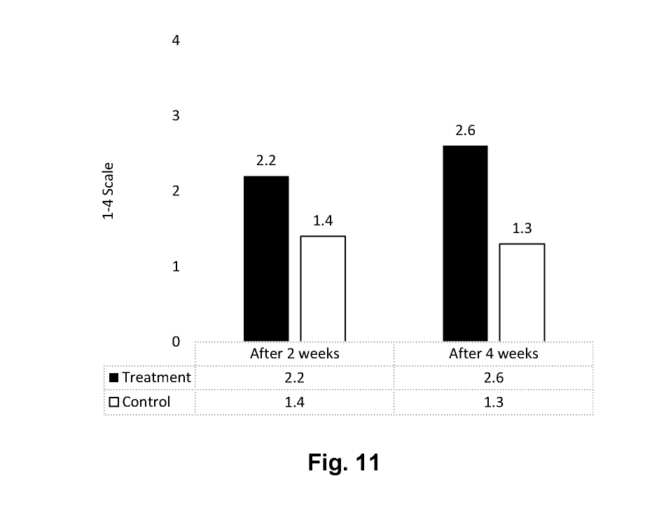

FIGS. 10 and 11 present a comparison between the treated area and the control area in the parameters of wrinkles around the eyes and firmness of the skin.

### Side Effects/Adverse Events

None of the volunteers reported of any side effects.

### CONCLUSIONS

On a test panel of 10 subjects who were included and completed the test after 4 weeks of use, the following conclusions were reached:
There was a significant improvement in wrinkles' condition around the eye at the treated side according to the analysis of the silicone imprints. The control area showed no significant change.
All the tested parameters that were assessed by the volunteers showed better results at the treated area when compared to the control area.
Seven out of 10 participants intend to purchase the product in the future.
No side effects were observed.

These results indicate that the product MW-001 is efficient for the treatment of rough and wrinkled skin, and is safe for use.

What is claimed is:

1. Method for treating or preventing pathological or non-pathological skin conditions, disorders or diseases comprising administering a conjugate of proline, hydroxyproline, or a salt thereof, conjugated with a fatty acid.

2. The method according to claim 1, wherein the fatty acid is omega-3 fatty acid, omega-6 fatty acid or omega-9 fatty acid.

3. The method according to claim 1, wherein the conjugate is a compound of Formula (I) 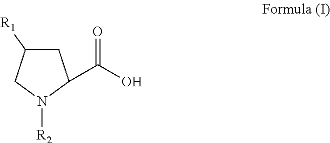
wherein R₁ is selected from hydrogen, unsaturated mono, poly and omega fatty acids, or OR₃;
R₂ and R₃ are independently selected from hydrogen or unsaturated mono, poly and omega fatty acids, provided that both R₁ and R₂ are not hydrogen and that if R₁ is OR₃, both R₂ and R₃ and not hydrogen.

4. The method according to claim 3, wherein R₁ is OR₃.

5. The method according to claim 1, wherein the conjugate is a conjugate of hydroxyproline and docosahexaenoic acid (DHA) according to formula MW-001:

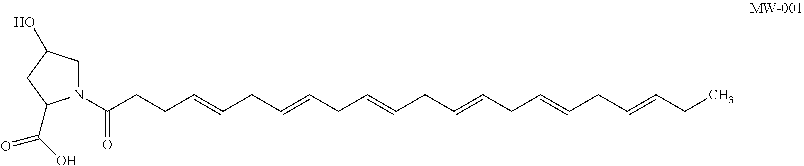 [SMILES: CC/C=C/C/C=C/C/C=C/C/C=C/C/C=C/C/C=C/CCC(=O)N1CC(O)CC1C(=O)O]

6. The method according to claim 1, wherein the skin condition is skin aging.

7. The method according to claim 1, wherein the skin condition, disorder or disease is acne, irritant diaper rash, irritant dermatitis, psoriasis, rosacea, dermatitis, impetigo, forunculosis, burns and wounds.

8. The method according to claim 1, wherein the conjugate is co-administered with an antibiotic.

9. The method according to claim 1, wherein a topical, dermatological or cosmetic formulation comprising said conjugate is administered.

10. The method according to claim 1, wherein an oral formulation comprising said conjugate is administered.

11. A method for reducing wrinkles comprising the step of administering a conjugate of proline, hydroxyproline, or a salt thereof, conjugated with a fatty acid.
